(19) United States
(12) Patent Application Publication
(10) Pub. No.: US 2025/0288793 A1
(43) Pub. Date: Sep. 18, 2025

(54) ASSEMBLY COMPRISING A COMPONENT FOR A BLOOD PUMP AND A PLUG

(71) Applicant: Berlin Heart GmbH, Berlin (DE)

(72) Inventor: Eike Schärff, Berlin (DE)

(73) Assignee: Berlin Heart GmbH, Berlin (DE)

(21) Appl. No.: 19/082,429

(22) Filed: Mar. 18, 2025

(30) Foreign Application Priority Data
Mar. 18, 2024 (EP) 24 164 107.5

(51) Int. Cl. A61M 60/878; A61M 60/178; A61M 60/205; A61M 60/414; H01R 13/627

(52) U.S. Cl. CPC A61M 60/878; A61M 60/178; A61M 60/205; A61M 60/414; H01R 13/627; H01R 2201/12

(57) ABSTRACT

An assembly comprising a component for a blood pump and a plug, wherein the component has a plug socket for detachably receiving the plug in order to establish an electrical connection to the blood pump. The plug may have a coupling portion arranged at a tip of the plug and insertable into the plug socket. The plug socket may comprise an electrically insulating socket housing having a housing inlet and a housing interior adjoining the housing inlet for receiving the coupling portion, wherein an alignment from the housing inlet into the housing interior defines an insertion direction of the plug. The plug socket may comprise a locking unit configured to detachably lock the plug inserted into the plug socket and configured to lock the plug in the socket housing without restricting rotation of the plug in the socket housing about the insertion direction.

### CROSS-REFERENCE TO RELATED APPLICATION

This application claims priority to EP Application No. 24 164 107.5, filed on Mar. 18, 2024, the entire content incorporated herein by reference.

### BRIEF DESCRIPTION OF THE DRAWINGS

Specific exemplary embodiments of the assembly formed of component and plug are described below with reference to the figures. An overview of what is shown in the figures is given first.

FIG. 1 shows an assembly formed of a component with a plug socket and a connection cable with a plug for establishing an electrical connection between the component and the blood pump;

FIG. 2 shows the plug and a cross-section through the plug socket from FIG. 1 in an enlarged view;

FIG. 3 shows a cross-section of the plug socket from FIG. 1 along an insertion direction in an enlarged view;

FIG. 4 shows an embodiment of the locking element as used in the plug socket from FIG. 1;

FIG. 5 shows the plug from FIG. 1 individually in an enlarged view;

FIGS. 6-8 show the plug and the plug socket from FIG. 1 in different phases during insertion and removal of the plug into and out of the plug socket;

FIG. 9 shows an alternative embodiment of a plug and a plug socket to FIG. 1;

FIG. 10 shows a locking element as used in the alternative embodiment of the plug socket from FIG. 9;

FIG. 11 shows the plug from FIG. 1 with an alternative embodiment of a plug socket to FIG. 1;

FIG. 12 shows the assembly 1 in conjunction with a blood pump.

The exemplary embodiments shown in the figures are described in detail below. In the following description and in the figures, identical features are provided with the same reference sign. For reasons of clarity, reference signs are not always given in each example, even if the associated feature is shown in the relevant figure.

### DETAILED DESCRIPTION

The invention relates to the field of medical technology, in particular cardiac support systems and electrical apparatus engineering.

Cardiac support systems with blood pumps to maintain blood circulation are known in medicine. Many such pumps, such as VAD pumps (Ventrical Assist Devices), are at least partially implantable and also transportable, so that people with such systems are at least partially mobile.

To control the blood pump and supply the blood pump with electrical energy, the blood pumps are usually connected to extracorporeal components such as a control unit. The electrical connection between the blood pump and the extracorporeal component is made via a connection cable (called a driveline). The connection cable is electrically and mechanically connected to the component via a detachable plug connection so that the component used may be replaced, for example due to a defect. Due to the vital function of the blood pump for the patient, increased requirements are placed on this plug connection. The plug connection must not only enable a secure electrical connection between the component and the blood pump. It must also include a mechanical safety device to prevent the connection cable from being accidentally disconnected from the component. In addition, the plug connection must be configured to be repeatedly connected and disconnected. At the same time, it must be ensured that the component is able to be cleaned and disinfected when plugged in.

A number of detachable connection systems are described.

US 2021/0361932 A1, for example, describes systems and methods for connections in a medical device system.

U.S. Pat. No. 10,953,145 discloses a connector assembly for connecting external power sources to an implanted medical device.

The problem addressed in this disclosure is that of providing an improved connection assembly between the blood pump and a component for the blood pump.

This problem is solved by the assembly described in this application.

In an example the assembly comprises a component for a blood pump and a plug, wherein the component has a plug socket for detachably receiving the plug in order to establish an electrical connection to the blood pump. The plug has a coupling portion that is arranged at the distal end of the plug and may be inserted into the plug socket. The plug socket for its part has an electrically insulating socket housing, which comprises a housing inlet and a housing interior adjoining the housing inlet for receiving the coupling portion, wherein an alignment from the housing inlet into the housing interior defines an insertion direction of the plug. In addition, the plug socket also has a locking unit which is configured to detachably lock the plug inserted into the plug socket, wherein the locking unit is configured to lock the plug in the socket housing without restricting rotation of the plug in the socket housing about the insertion direction.

The plug and plug socket form a connection assembly. As described above, the locking unit is configured to hold the plug inside the plug socket, preferably mechanically, in order to prevent the plug from being accidentally pulled out of the plug socket, thereby increasing the operating safety of the connection assembly. The formulation that the locking unit is configured to lock the plug in the socket housing without restricting the rotation of the plug means that the plug may be rotated by any angular range around the insertion direction of the plug in the plug socket.

The inventors have recognized that a previously unsolved medical problem is the prevention of so-called driveline infections. A driveline infection is an infection of the tissue around a driveline exit site on the patient's body. Driveline infection is favored if the connection cable is exposed to many movements such as rotation and torsion. Through their work, the inventors have realized that rotations and torsions of the connection cable may be reduced if the plug of the connection cable may be rotated about the insertion direction as desired when plugged in and locked. Thus, when the patient handles the component for the blood pump, the plug in the plug socket may rotate in accordance with a relative movement of the component relative to the patient's body, so that rotation or torsion of the cable is reduced at the exit point of the connection cable into the patient's body. At the same time, operating safety is maintained thanks to the mechanical locking of the plug within the plug socket.

Further possible embodiments of the assembly are described below.

In one embodiment, the component may be a control unit that is configured to output control signals via the plug socket and/or to provide electrical energy to operate the blood pump. Alternatively, the component may also be a power supply unit that is configured to provide electrical energy to operate the blood pump via the plug socket.

In another embodiment, the coupling region of the plug may be rotationally symmetrical with respect to the insertion direction. The rotationally symmetrical configuration is a simple design option that may simplify the production of the plug. Furthermore, friction may thus be reduced when the plug rotates in the plug socket.

In one embodiment, the coupling portion of the plug may comprise a, preferably electrically insulated, locking portion. The locking unit may further have a locking element, wherein the locking element is displaceable between a locking position and a non-locking position and, in the locking position, projects partially into an alignment of the housing inlet and is configured to engage in the locking portion of the plug when the plug is inserted in order to counteract the plug being pulled out. There are various ways of ensuring that the plug rotates in the plug socket while simultaneously locking it in place. This realization of the locking unit may be particularly suitable for counteracting accidental removal of the plug from the plug socket.

In a variant of this embodiment the locking portion may additionally have one or more lateral projections which at least partially surround the coupling portion in a plane perpendicular to the insertion direction and are arranged in such a way that, when the plug is inserted, these are engaged behind by the locking element in the locking position. The one or more projections should be arranged around the coupling portion in such a way that the projections are also engaged behind by the locking element when the plug is rotated relative to the plug socket. The one or more projections may, for example, be exactly one projection that runs in a ring around the coupling portion.

In another variant of this embodiment the locking portion may have a bearing surface which surrounds the coupling portion at least in portions with respect to the insertion direction of the plug and which is arranged on the side of the projection or of the plurality of projections remote from the plug tip. This may be advantageous in order to reduce friction of the locking element in the region of the locking portion and thus facilitate rotation of the plug in the plug socket. The contact surface may extend over the entire circumference of the coupling portion. Alternatively, the contact surface may also be interrupted in portions, i.e. have one or more recesses, for example. However, the recesses should be configured in such a way that the rotatability of the locked plug in the plug socket is maintained and, for example, the locking element may neither fully nor partially engage in the one or more recesses.

In one variant of this embodiment, for example, the contact surface and projection may be configured as part of a groove surrounding the coupling portion with respect to the insertion direction.

In a further variant of this embodiment, the locking element may have a plate-shaped portion which is arranged transversely, preferably perpendicularly, to the insertion direction, and comprises a locking edge, wherein the locking edge for engaging in the locking region of the plug has a portion with a circular arc-shaped profile which, in the locking position of the locking element, projects into the alignment of the housing inlet. The circular arc-shaped profile of the locking edge may reduce the force required to rotate the plug in the plug socket. The circular arc-shaped profile of the edge may preferably be concave with respect to the locking bar, i.e. a connecting section between two points of the circular arc-shaped profile does not run through the locking bar. In one realization, the circular profile may additionally or alternatively have an arc portion of at least 120°, at least 160° or at least 180°.

In another realization, the locking edge may be an outer edge of the plate-shaped portion. The locking edge may, for example, be formed by a U-shaped recess in the plate-shaped portion for the plug to pass therethrough. Alternatively, the plate-shaped portion may comprise a through-hole for the plug to pass therethrough and the circular arc-shaped locking edge may be formed by an edge of the through-hole. The through-hole may be oval in shape and the locking edge may have two semi-circular portions with the same radius, which are connected to each other by two straight portions.

In another realization of the locking element with a plate-shaped portion, the plate-shaped portion may comprise an end face which is aligned in the direction of the housing inlet, and a transition between the end face and the locking edge may have a chamfer. The chamfer may facilitate the insertion of the plug into the plug socket by enabling or simplifying a temporary displacement of the locking element from the locking position into the non-locking position by insertion of the plug.

In another variant of the embodiment, the locking unit may additionally or alternatively comprise a resetting unit which is configured to fix the locking element in the locking position when no external force is applied.

In a further embodiment, the component may have an actuating element that is connected to the locking unit, and the locking unit may be configured to release the locking when the actuating element is actuated. In a variant of this embodiment, which also has the resetting unit, the locking unit may be configured to counteract a force generated by the resetting unit when the actuating element is actuated in order to move the locking bar into the non-locking position. This may be advantageous to give a patient or other user of the component a convenient way to release the locking of the plug in the plug socket.

In another embodiment one or more electrical contact elements for establishing an electrical connection between the component and the plug may be arranged in the coupling portion and within the housing interior.

In a variant of this embodiment, the one or more electrical contacts may be arranged inside the plug socket between the housing inlet and the locking unit or on a side of the locking unit remote from the housing inlet. An assembly of locking portion and electrical contacts along the coupling portion of the plug may be realized accordingly.

In another variant of this embodiment, the electrical contact elements of the plug may be arranged on an outer side of the coupling portion and the contact elements of the plug socket may be arranged in a, preferably partially cylindrical, portion of the housing interior. Alternatively, the coupling portion of the plug may be configured in the form of a hollow cylinder with a hollow cylinder opening in the insertion direction and the contact elements of the plug may be arranged inside the hollow cylinder. In this case, the plug socket comprises a pin, on the surface of which the contact elements of the plug socket are arranged.

In another variant, the contact elements may be arranged one behind the other along the insertion direction on or in the plug and in the plug socket. In addition, the contact elements may either be configured as contact surfaces on the plug and as spring-loaded contact pick-ups on the plug socket, or vice versa. Spring-loaded contact pick-ups may be annular spring contacts or pin spring contacts, for example.

In a variant of this embodiment, the plug may comprise a plug sealing element in the coupling portion and a plug socket sealing element may be arranged between the housing inlet and the one or more electrical contact elements of the plug socket, which plug socket sealing element is configured to co-operate with the plug sealing element when the plug is inserted in order to counteract the penetration of liquid and/or particles into the housing interior. The sealing element of the plug may also be configured as a ring seal, for example, and the sealing element of the plug socket as a sealing contact surface or vice versa.

In one realization of this variant, the sealing element of the plug socket may be arranged between the housing inlet and the locking unit. This may help to protect not only the electrical contact elements but also the locking unit from particles and liquids.

In a further embodiment, in order to limit insertion of the plug into the socket housing, the housing interior may comprise a stop surface which is aligned in the direction of the housing inlet, and the plug may comprise at least one lateral projection which has an end face which is aligned in the direction of the tip of the plug and which bears against the stop surface when the plug is inserted. The stop surface may, for example, be oriented transverse to the insertion direction.

In a variant of this embodiment, for example, the stop surface may be realized by an interface between two adjacent portions of the housing interior with different diameters, wherein the portion with the larger diameter is arranged closer to the housing inlet. The projection of the plug may be the same projection as the projection of the locking region.

Alternatively or additionally, the plug may also have an optically visible marking on the stop surface, which may be used to indicate whether the plug has been inserted sufficiently far into the plug socket.

In a variant of this embodiment, the end face of the projection may have a chamfer that at least partially surrounds the end face laterally. This may make it easier to insert the plug into the plug socket.

In another embodiment of the assembly, the assembly may comprise a blood pump and a connection cable which comprises the plug and by which the blood pump may be electrically connected to the component.

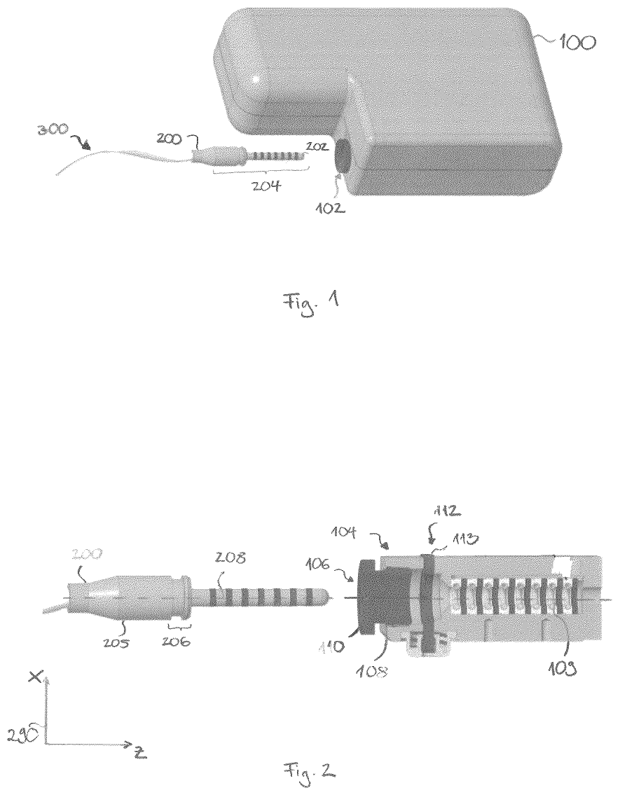

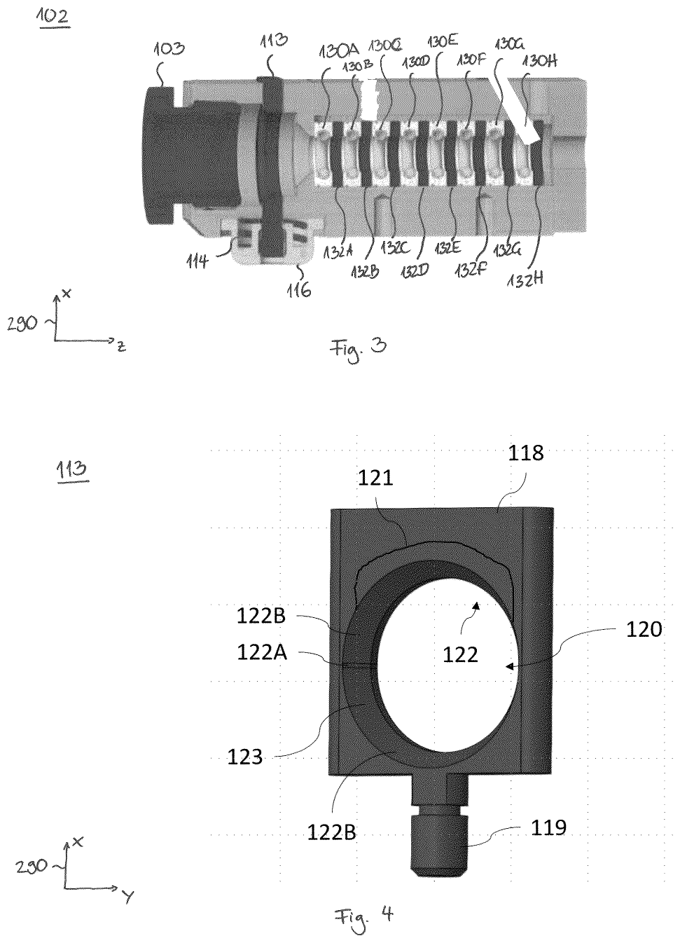

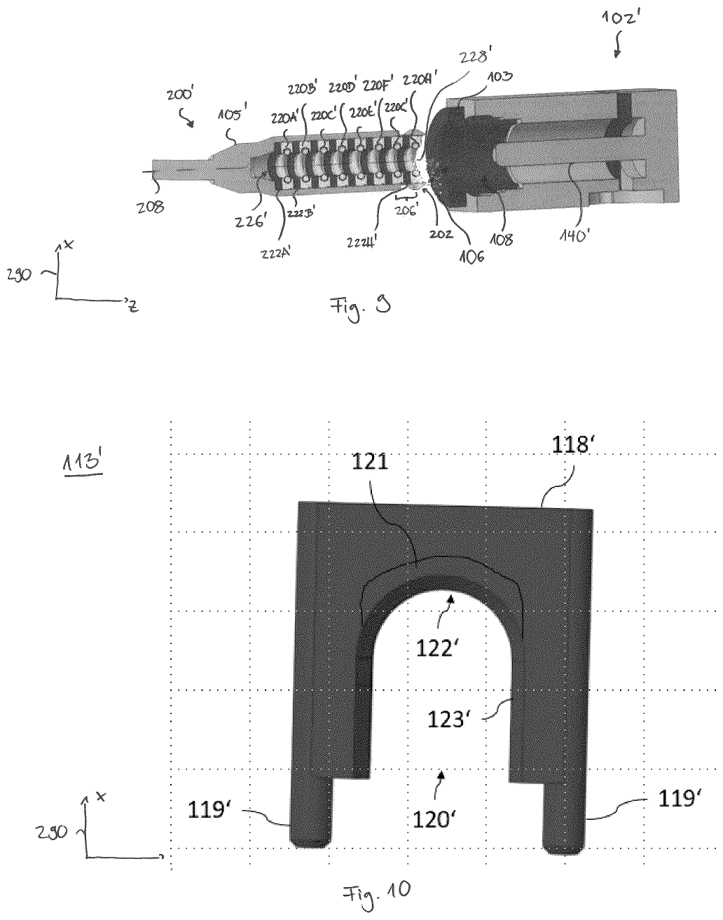

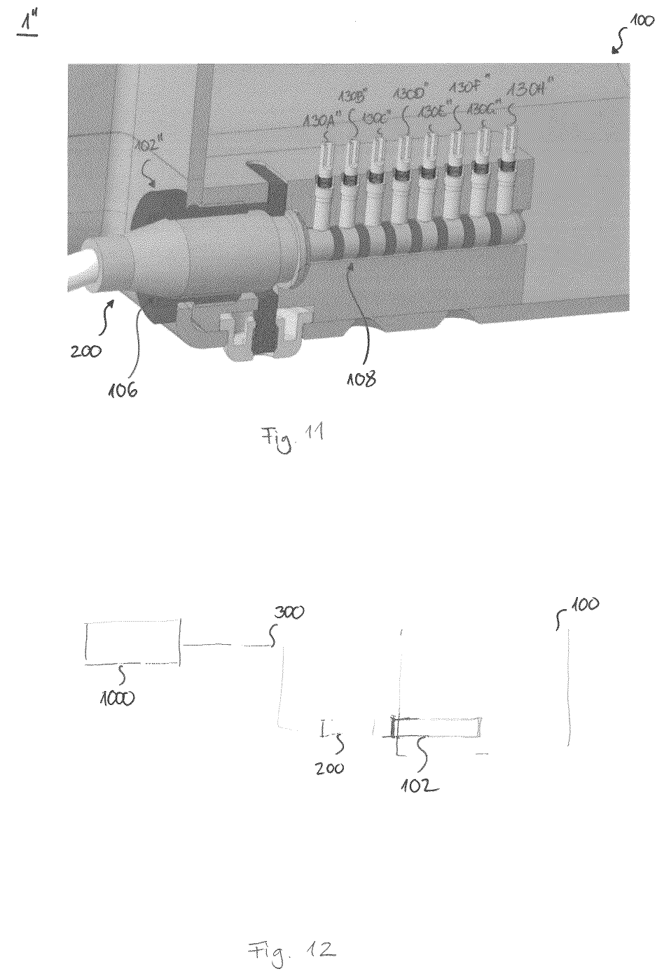

A first exemplary embodiment of the assembly is described with reference to FIGS. 1 to 8. Two alternative exemplary embodiments are described with reference to FIGS. 9 and 10 and with reference to FIG. 11. The assembly from FIGS. 1 to 8 is described with reference to FIG. 12 in conjunction with a blood pump. With regard to the first exemplary embodiment, an overview of the first exemplary embodiment of the assembly is given with reference to FIGS. 1 and 2.

FIG. 1 shows an assembly 1 comprising a component 100 with a plug socket 102 and a connection cable 300 with a plug 200 for establishing an electrical connection between the component 100 and a blood pump. FIG. 2 shows the plug 200 and a cross-section through the plug socket 102 from FIG. 1 in an enlarged view.

In the exemplary embodiment shown, the component 100 is a control unit for the blood pump and is configured to supply a blood pump with electrical energy via the plug socket 102 and the connection cable 300 with the plug 200. At the same time, operating parameters for operating the blood pump may be selected via input interfaces of the control unit 100 not shown in the figures. Furthermore, the control unit 100 is configured to transmit control signals to the blood pump via the plug socket 102 in accordance with the selected operating parameters.

The plug 200 comprises a coupling portion 204 arranged at its distal end or the tip 202 of the plug 200 and insertable into the plug socket 102. A mechanical as well as an electrically conductive connection between the plug 200 and the plug socket 102 may be established via the coupling portion 204. The plug socket 102 is configured to detachably receive the plug 200 and for this purpose has an electrically insulating socket housing 104, which comprises a housing inlet 106 and a housing interior 108 adjoining the housing inlet 106 for receiving the coupling portion 204. An alignment of the housing inlet 106 into the housing interior 108 defines an insertion direction 110 of the plug 200 into the plug socket 102. The insertion direction 110 coincides with a longitudinal axis 109 of the housing interior 108 for the plug socket 102 and with a longitudinal axis 208 of the plug 200 for the plug 200. For a better description of extensions and arrangements of components of the assembly 1 across the individual figures, a coordinate system 290 is also drawn in FIG. 2, the z-axis of which runs parallel to the insertion direction 110.

The plug socket 102 furthermore comprises a locking unit 112 which is configured to detachably lock the plug 200 inserted into the plug socket 102. The locking unit 112 is configured here to lock the plug 200 in the socket housing 104 without restricting rotation of the plug 200 in the socket housing 104 about the insertion direction 110.

The formulation that the rotation of the plug 200 in the plug socket 102 about the insertion direction 110 is not restricted means that the rotation of the plug 200 within the plug socket 102 is not hindered by an end stop. In the exemplary embodiment shown, the plug 200 may be rotated through any angular range. However, the term “restriction” does not mean that no friction occurs during rotation between the plug 200 and the plug socket 102. This is also provided in the exemplary embodiment shown. However, the friction does not restrict the rotation of the plug 200 in principle, but merely leads to greater force being required to rotate the plug 200 in the plug socket 102.

There are various ways of forming the locking unit 112 and the plug 200 in order to ensure rotation of the plug 200 in the socket housing 104. In the exemplary embodiment of the assembly 1 shown in FIGS. 1 to 8 this is achieved in that the coupling portion 204 of the plug 200 comprises an electrically insulated locking portion 206 and the locking unit 112 has a locking element 113, wherein the locking element 113 is displaceable between a locking position and a non-locking position and, in the locking position, projects partially into the alignment of the housing inlet 106 and is configured to engage in the locking portion 206 of the plug 200 when the plug 200 is inserted in order to counteract the plug 200 being pulled out. Details of the locking portion 206 of the plug 200 and the locking unit 112 are described below with reference to FIGS. 3 to 8. First of all, FIGS. 3 to 5 will be discussed.

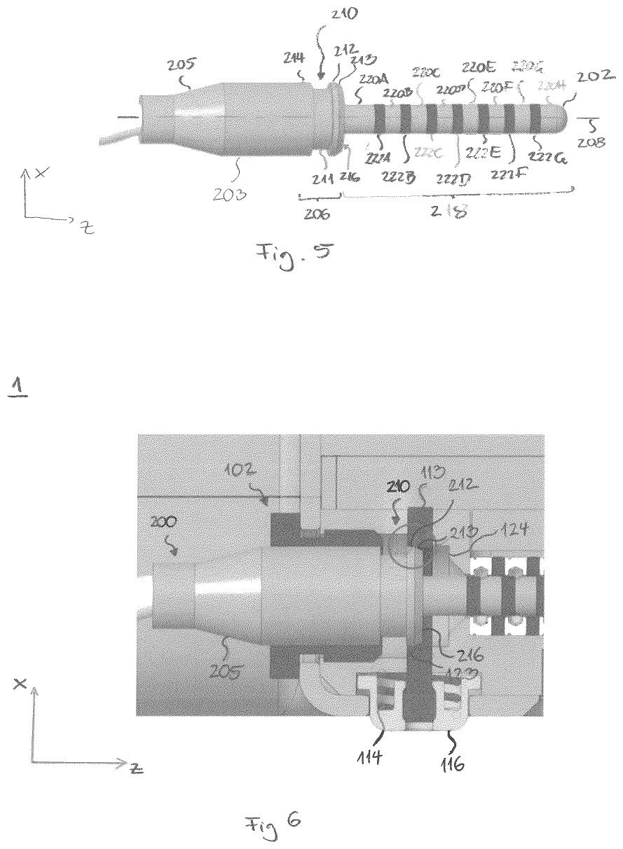

FIG. 3 shows a cross-section of the plug socket 102 from FIG. 1 along the insertion direction 110 in an enlarged view. FIG. 4 shows an exemplary embodiment of the locking element 113 as used in the plug socket 102 from FIG. 1. FIG. 5 shows the plug 200 from FIG. 1 individually in an enlarged view.

Firstly, with reference to FIGS. 3 and 4, the locking unit 112 of the plug socket 102 is explained in more detail. The locking unit 112 has a locking element 113. The locking element 113 is displaceable between a locking position and a non-locking position, wherein the locking element 113 in the locking position projects partially into the alignment of the housing inlet 106 and is configured to engage in the locking portion 206 of the plug 200 when the plug 200 is inserted in order to counteract the plug 200 being pulled out. In the locking position, an upper region of the locking element 113 protrudes into the alignment of the housing inlet 106. In order to be moved from the locking position to the non-locking position, the locking element 113 must be displaced in the positive x-direction. In the non-locking position, no part of the locking element 113 protrudes into the alignment of the housing inlet 106 due to a through-hole.

In the exemplary embodiment shown in FIGS. 1 to 8 the locking unit 112 further has a spring 114 as resetting unit which is configured to fix the locking element 113 in the locking position when no external force is applied. The component furthermore has an actuating element 116 which is connected to the locking unit 113. The locking unit 113 is further configured to release the locking when the actuating element 116 is actuated. In the exemplary embodiment shown, the spring 114 is connected to the locking element 113 in such a way that the spring permanently acts with a resetting force in the negative x-direction, so that the locking element 113 is held in the locking position without external force or is brought back into the locking position by the spring 114 after the locking element 113 has been moved into the non-locking position. Furthermore, the spring 114 is arranged within the actuating element 116. In this exemplary embodiment, the actuating element 116 is a mechanical button. By applying a force in the positive x-direction to the actuating element 116, the spring 114 is compressed and at the same time the locking element 113 is displaced in the positive x-direction from the locking position to the non-locking position. This allows the plug 200 to be released from the plug socket 102. In this exemplary embodiment, the spring 114 has a dual function: returning the locking element 113 to the locking position and returning the actuating element 116 to an initial position. However, the assembly of locking element, resetting unit and actuating element shown is only an example. The actuating element could, for example, be realized with an electrical component instead of a purely mechanical one, so that, for example, the actuating element is not mechanically connected to the resetting unit and the locking element.

In FIG. 4, the locking element 113 is shown individually in a front view, i.e. viewed along the positive z-axis. The locking element 113 in the exemplary embodiment of the assembly 1 has a plate-shaped portion 118 which, when the locking element 113 is installed in the plug socket 102, is arranged perpendicular to the insertion direction, i.e. in the x-y plane. The locking element 113 further comprises a pin 119 for mechanical force transfer between the actuating element 116 and the locking element 113. Furthermore, the locking element 113 comprises a through-hole 120, the edge region of which forms a locking edge 122. The locking edge 122 is formed from two opposing semicircular portions 122B with the same radius, which are connected to one another by two likewise opposing straight portions 122A. The through-hole 120 is used to feed through part of the coupling region 204 of the plug 200. In the locking position of the locking element 113, an upper region 121 of the circular arc-shaped locking edge 122 projects into the alignment of the housing inlet 106 and serves to engage the locking unit 112 in the locking region 206 of the plug 200. This is explained in greater detail below with reference to FIG. 5.

FIG. 5 shows an enlarged view of the plug 200. As is visible in FIG. 5, the locking portion 206 is arranged in a grip element 205 of the plug 200. The grip element 205 may be made of an electrically insulating material. In the exemplary embodiment shown, the grip element 205 is made of a polymer. The locking portion 206 is arranged in an end of the grip element 205 pointing towards the plug tip 202 and comprises a groove 210 which is made in the grip element 205 and which runs all the way around the plug 200 symmetrically to the longitudinal axis 208. Lateral edges of the groove 210 form two lateral projections 212 and 214, which run around the coupling portion 204 in a plane perpendicular to the insertion direction 110. The groove 210 is arranged here in such a way that the region 121 of the locking edge 122 of the locking element 113 engages in the groove 210 in the locking position and in so doing engages behind the projection 212. A surface 211 formed in the recess of the groove further forms a support surface for the locking edge 122. In this example, the entire plug 200 is rotationally symmetrical with respect to the insertion direction 110. However, this is not a mandatory requirement. For example, instead of the circumferentially arranged lateral projection 212, a plurality of spaced-apart lateral projections may also be circumferentially arranged. The only requirement here is that, despite the spacing of the projections, it is ensured that at least one projection is engaged behind uninterruptedly by the locking element even when the plug 200 rotates in the plug socket 102.

In the following, the interaction between the plug 200 and the plug socket 102 during the insertion of the plug 200 into the plug socket 102 is described in more detail with reference to FIGS. 6 to 8.

FIGS. 6 to 8 show the plug 200 and the plug socket 102 from FIGS. 1 to 5 in different phases during insertion and removal of the plug 200 into and out of the plug socket 102.

FIG. 6 shows one phase of the insertion of the plug 200 into the plug socket 102. In the phase shown in FIG. 1, the projection 212 of the plug 200 is pushed straight through the opening 120 of the locking element 113. It is visible here how the chamfer 213 on the end face 216 of the grip element 205 of the plug 200 interacts with the chamfer 123 of the locking element 113 in order to move the locking element 113 from the locking position to the non-locking position in the z-direction when the plug 200 is inserted, so that the lateral projection 212 may pass through the through-hole 120 of the locking element 113.

As soon as the projection 112 has passed through the through-hole 120, the locking element 113 due to the resetting force of the spring 114 is returned to the locking position, in which the upper region 121 of the locking edge 122 engages in the groove 210. This is shown in FIG. 7. The engagement behind the projection 212 by the upper region 121 of the locking edge 122 counteracts a pulling of the plug 200 out from the plug socket 102. The plug 200 is unable to be accidentally pulled out of the plug socket 102. FIG. 6 also shows how the end face 216 of the plug 200 co-operates with a stop surface 124 of the plug socket to limit the insertion of the plug 200 into the plug socket 102 in the insertion direction 110. The stop surface 124 is an interface between two adjacent, approximately cylindrical regions of the housing interior 108 with different radii.

The lateral projection 212 therefore has a dual function. On the one hand, the projection 212 together with the locking element 113 counteracts accidental removal of the plug 200 from the plug socket 102. Secondly, the end face 216 of the lateral projection 212 limits the insertion of the plug 200 into the plug socket 102, so that there is no risk of insertion of the plug 200 causing damage to the housing interior 108. In addition, the end face 216 and stop surface 124 are arranged relative to the locking element 113 and groove 210 in such a way that when the plug 200 is fully inserted, the locking element 113 may engage in the groove 210 of the plug 200.

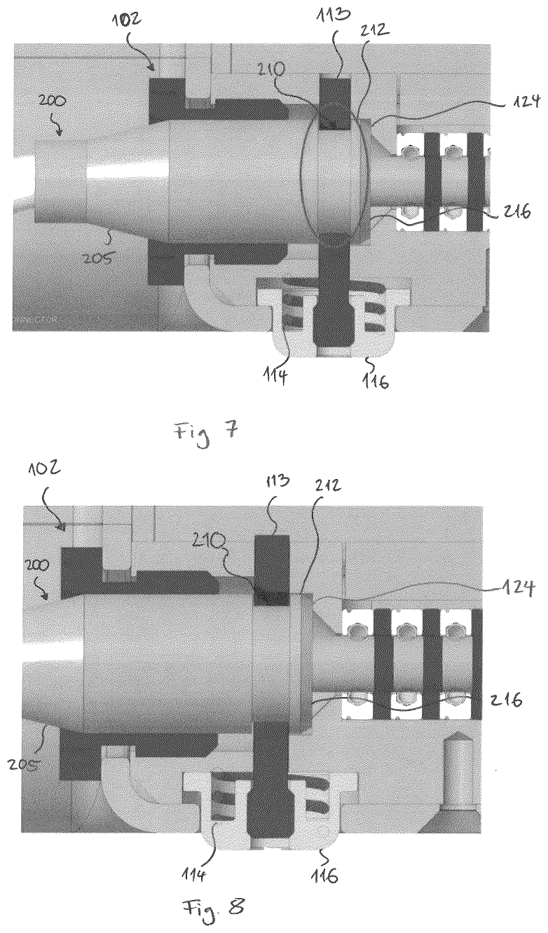

Finally, FIG. 8 shows a first phase when the plug 200 is pulled out of the plug socket 102. If the plug 200 is to be pulled out of the plug socket 102, the locking element 113 of the plug socket 102 must first be moved from the locking position to the non-locking position. This may be done by pressing on the actuating element 116. The pressure must be strong enough that it overcomes the resetting force of the spring 114 and the locking element 113 is displaced in the z-direction to such an extent that the locking edge 122 no longer engages in the groove 210 of the plug 200. This phase is shown in FIG. 8. The plug 200 may then be removed from the plug socket 102 by moving it in the opposite direction to the insertion direction 110.

Now that the mode of operation of the locking unit 112 has been described in detail, the establishment of the electrical connection between the plug 200 and the plug socket 102 is described below. Reference is once again made to FIGS. 3 and 5.

In FIG. 3 it may be seen that the plug socket 102 has a total of 8 contact elements 130A-H, which are configured as annular spring contacts and are arranged one behind the other along the insertion direction in a rear region of the housing interior 108. The annular spring contacts 130A-H are electrically insulated from one another by electrically insulating ring elements 132A-H, wherein an insulating ring element is always arranged between two annular spring contacts arranged next to one another. FIG. 5 shows that in a contact portion 218 of the coupling portion 204 located at the plug tip 202, a number of contact surfaces 220A-H are arranged along the insertion direction 110 of the plug 200 and are electrically insulated from each other by electrically insulating ring elements 222A-G. The contact surfaces 220A-H are arranged here along the plug 200 in such a way that when the plug is fully inserted, an electrical connection is realized between one of the annular spring contacts 130A-H and one of the contact surfaces 220A-H. There are 8 contact elements in the example. However, the number of contact elements may be freely varied as required. In the example shown, the locking unit 112 is arranged in the plug socket 102 between the housing inlet 106 and the ring contacts 130A-H. However, as will be shown later using another example, this arrangement is not absolutely necessary.

It will also be explained below with reference to FIGS. 3, 5 and 7 how the use of sealing elements may counteract the penetration of liquids and dirt particles. A sealing lip 103 is provided in the plug socket 102 for this purpose and also forms the housing inlet 106. In turn, the plug 200 comprises a sealing surface 203, which is located on a side of the grip element 205 remote from the plug tip 208. The sealing lip 103 of the plug socket 102 is molded from a soft plastic and, as shown in FIG. 7, rests on the sealing surface 203 of the plug 200 when the plug 200 is inserted. The sealing lip 103 and sealing surface 203 work together here to minimize the penetration of dirt and liquid into the housing interior 108. In other exemplary embodiments, however, it is also possible to use a ring seal instead of the sealing lip 103. In addition or alternatively, the sealing lip and sealing surface may also be arranged the other way around on the plug and plug socket.

In the last part of this disclosure, reference is also made to FIGS. 9 to 11 to describe further alternative exemplary embodiments of the component and the plug. First of all, an initial alternative embodiment is described on the basis of FIGS. 9 and 10.

FIG. 9 shows an alternative embodiment to FIG. 1 of a plug 200′ and a plug socket 102′ for a component. The plug 200′ and plug socket 102′ are shown in FIG. 9 in a cross-section along the insertion direction 110 of the plug 200′ into the plug socket 102′. FIG. 10 shows a locking element 113′ as used in the alternative embodiment of the plug socket 102′ from FIG. 9.

The plug 200′ comprises a grip element 105′, which is elongate and may be inserted into the plug socket 102′ in the z-direction, i.e. along its longitudinal axis of the plug 200′. In contrast to the plug 200, however, the grip element 105′ is hollow-cylindrical along the longitudinal axis 208 and comprises a cavity opening 228′, which is adjoined by a cavity 226′. A total of 8 contact elements 220A′-H′ in the form of annular springs are arranged within this cavity, wherein each two neighboring annular springs 220A′-H′ are separated from each other by an electrically insulating ring 222A′-H′. Furthermore, the plug 200′ also comprises a locking portion 206′. However, this is not arranged at an end of the grip element 205′ remote from the plug tip 202, as is the case with the plug 200, but is located in the immediate vicinity of the plug tip 202.

The plug socket 102′, like the plug socket 102, comprises the housing inlet 106, which is adjoined by a housing interior 108. As with the plug socket 102, the housing inlet 106 is formed by the sealing element 103. Furthermore, the plug socket 102′ also comprises a locking unit, of which only the locking element 113′ is shown in FIG. 8. In contrast to the plug socket 102, the locking element 113′ of the plug socket 102′ is arranged at an end of the housing interior 108 remote from the housing inlet 106. For the assembly of the contact elements compatible with the plug 200′, a pin 140′ is arranged within the housing interior 108 and is visible in cross-section in FIG. 8. On a side of the pin 140′ facing away from the viewer, and therefore not visible in FIG. 8, 8 contact elements are arranged in the form of contact surfaces which, when the plug 200′ is inserted, establish an electrically conductive connection between the plug 200′ and the plug socket 102′ with the ring elements 220A′-H′. In the following, the locking element 113′ is described in more detail with reference to FIG. 10.

FIG. 10 shows the locking element 113′ in the x-y plane, i.e. in a plane perpendicular to the insertion direction 110. Furthermore, the locking element 113′ is shown from the perspective of a plug 200′ to be inserted, i.e. looking in the positive z-axis.

Like the locking element 113, the locking element 113′ also comprises a plate-shaped portion 118′, which, however, does not comprise a through-hole, but a U-shaped recess 120′. The U-shaped recess 120′ is provided within the locking element 113′ in such a way that it also has the upper region 121, which is formed as a circular arc-shaped edge region 122′ of the recess 120′ and is arranged in such a way that it engages in the groove 110 of a plug 200′ when the plug 200′ is inserted. Furthermore, like the locking element 113, the locking element 113′ comprises a chamfer 123′ at the transition between the plate-shaped portion 118′ and the edge region 122′ in order to facilitate insertion of the plug 200′ into the plug socket 102′. Furthermore, the locking element 113′ comprises two pins 119′ for transmitting force between the locking element 113′ and the actuating element.

Finally, a further alternative embodiment of the plug socket is described below with reference to FIG. 11.

FIG. 11 shows the plug 200 from FIG. 1 with a further alternative embodiment of a plug socket 102″ to FIG. 1.

The plug 200 shown in FIG. 11 is identical to the plug shown in FIG. 1 and is therefore not described further here. Furthermore, a plug socket 102″ shown in FIG. 11 is also largely identical to the plug socket 102 shown in FIG. 1. The only difference between the plug socket 102 and the plug socket 102″ is that the plug socket 102″ has pin spring contacts 130A″-130H″ instead of the annular spring contacts 130A-H. The pin spring contacts 130A″-130H″ are arranged in such a way that they press on the contact surfaces 220A-H of the plug 200 in the negative x-direction when the plug 200 is inserted. In other exemplary embodiments, however, the contact pins may also be oriented differently. Furthermore, it is also possible for a contact surface at the tip of the plug 200 to be removed via a contact pin arranged in the insertion direction 110.

The last figure shows the use of assembly 1 in conjunction with a blood pump 1000. The blood pump 1000 may be electrically connected to the plug socket 102 of the component 100 via the connection cable 300, which comprises the plug 200.

To summarize, this disclosure describes an assembly (1) comprising a component (100) for a blood pump and a plug (200), wherein the component (100) has a plug socket (102) for detachably receiving the plug (200) to establish an electrical connection to the blood pump. The plug (200) has a coupling portion (204) arranged at a tip (202) of the plug (200) and insertable into the plug socket (102). Furthermore, the plug socket (102) comprises an electrically insulating socket housing (104), which has a housing inlet (106) and a housing interior (108) adjoining the housing inlet (106) for receiving the coupling portion (204), wherein an alignment from the housing inlet (106) into the housing interior (108) defines an insertion direction (110) of the plug (200). In addition, the plug socket (102) comprises a locking unit (112) which is configured to detachably lock the plug (200) inserted into the plug socket (102), wherein the locking unit is configured to lock the plug (200) in the socket housing (104) without restricting rotation of the plug (200) in the socket housing (104) about the insertion direction (110).

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

1. An assembly comprising:
a component for a blood pump; and
a plug;
wherein the component has a plug socket for detachably receiving the plug in order to establish an electrical connection to the blood pump;
wherein the plug has a coupling portion arranged at a tip of the plug and insertable into the plug socket; and
wherein the plug socket has:
an electrically insulating socket housing having a housing inlet and a housing interior adjoining the housing inlet for receiving the coupling portion, an alignment from the housing inlet into the housing interior defining an insertion direction of the plug, and
a locking unit configured to detachably lock the plug inserted into the plug socket;
wherein the locking unit is configured to lock the plug in the socket housing without restricting rotation of the plug in the socket housing about the insertion direction.

2. The assembly of claim 1, wherein:
the coupling portion of the plug comprises an electrically insulated locking portion, and
the locking unit has a locking element, wherein the locking element is displaceable between a locking position and a non-locking position and, in the locking position, projects partially into the alignment of the housing inlet and is configured to engage in the locking portion of the plug when the plug is inserted in order to counteract the plug being pulled out.

3. The assembly of claim 2, wherein the locking portion has one or more lateral projections at least partially surrounding the coupling portion in a plane perpendicular to the insertion direction, each of the one or more lateral projections arranged in such a way that, when the plug is inserted, the one or more lateral projections are engaged behind by the locking element in the locking position.

4. The assembly of claim 3, wherein the locking portion has a bearing surface surrounding the coupling portion with respect to the insertion direction of the plug and arranged on the side of the one or more lateral projections facing away from the plug tip.

5. The assembly of claim 2, wherein:
the locking element has a plate-shaped portion arranged transversely to the insertion direction, and comprises a locking edge; and
the locking edge has a portion with a circular arc-shaped profile which, in the locking position of the locking element, projects into the alignment of the housing inlet.

6. The assembly of claim 5, wherein the locking edge is an outer edge of the plate-shaped portion.

7. The assembly of claim 5, wherein:
the plate-shaped portion comprises a through-hole for passing the plug therethrough, and
the circular arc-shaped locking edge is formed by an edge of the through hole.

8. The assembly of claim 5, wherein the plate-shaped portion comprises an end face oriented in the direction of the housing inlet, a transition between the end face and the locking edge having a chamfer.

9. The assembly of claim 2, wherein the locking unit comprises a resetting unit configured to fix the locking element in the locking position when no external force is applied.

10. The assembly of claim 1, wherein:
the component has an actuating element connected to the locking unit, and
the locking unit is configured to release the locking when the actuating element is actuated.

11. The assembly of claim 1, wherein one or more electrical contact elements configured to establish an electrical connection between the component and the plug are arranged in the coupling portion and within the housing interior.

12. The assembly of claim 11, wherein:
the plug comprises a plug sealing element in the coupling portion, and
a plug socket sealing element is arranged between the housing inlet and the one or more electrical contact elements of the plug socket and is configured to co-operate with the plug sealing element when the plug is inserted in order to counteract the penetration of liquid and/or particles into the housing interior.

13. The assembly of claim 1, wherein:
the housing interior is configured to limit insertion of the plug into the socket housing and comprises a stop surface oriented towards the housing inlet, and
the plug comprises at least one lateral projection having an end face aligned in the direction of the tip of the plug, the end face bearing against the stop surface when the plug is inserted.

14. The assembly of claim 13, wherein the end face of the projection has a chamfer.

15. The assembly of claim 1, comprising a blood pump and a connection cable comprising the plug and by which the blood pump may be electrically connected to the component.
